(19) United States
(12) Patent Application Publication
(10) Pub. No.: US 2026/0234577 A1
(43) Pub. Date: Aug. 13, 2026

(54) INTEGRASE VARIANTS FOR GENE INSERTION IN HUMAN CELL

(71) Applicant: APPLIED STEMCELL, INC., Milpitas, CA (US)

(72) Inventors: Yanru Ruby TSAI, San Jose, CA (US); Alfonso FARRUGGIO, Stanford, CA (US)

(21) Appl. No.: 19/154,374

(22) PCT Filed: Feb. 7, 2024
(86) PCT No.: PCT/US2024/014900

§ 371 Date: Aug. 6, 2025

Related U.S. Application Data
(60) Provisional application No. 63/483,752, filed on Feb. 7, 2023.

(51) Int. Cl. C12N 9/12; C12N 9/22; C12N 15/11; C12N 15/90

(52) U.S. Cl. CPC C12N 9/1241; C12N 9/226; C12N 15/111; C12N 15/907; C07K 2319/09; C07K 2319/85; C12N 2310/20; C12Y 207/07

(57) ABSTRACT

Disclosed herein is a polypeptide comprising a variant of phiC31 integrase linked to a gRNA binding domain wherein the polypeptide recognizes a specific sequence in human genome. Also disclosed are compositions containing the polypeptide or the nucleic acid encoding the polypeptide, and methods of using the compositions, for inserting a transgene to the locus in human genome that contains the specific sequence.

### CROSS-REFERENCE TO RELATED APPLICATIONS

This application claims priority to U.S. provisional patent application No. 63/483,752, filed Feb. 7, 2023, the disclosure of which is incorporated herein by reference.

### FIELD OF THE INVENTION

The present invention generally relates to compositions and methods used for integrating transgenes into the genome of a cell.

### SEQUENCE LISTING

An official copy of the sequence listing is submitted concurrently with the specification electronically via EFS-Web as an ASCI I formatted sequence listing with a file name of 044903-8032WO01_ST26, a creation date of Feb. 7, 2024, and a size of 44,158 bytes. The sequence listing contained in this ASCII formatted document is part of the specification and is herein incorporated by reference in its entirety.

### BACKGROUND OF THE INVENTION

RNA-guided Cas9 nucleases derived from clustered regularly interspaced short palindromic repeats (CRISPR)-Cas systems have provided a versatile tool for editing the genome of diverse organisms. However, current technologies based on CRISPR-Cas system have limited ability of inserting large DNA fragments and are unable to perform homology-based editing such as targeted transgene insertion in non-dividing cells (e.g., neurons) or in cells with DNA homologous recombination deficiency. Therefore, there remains a need for new genome engineering technologies that are affordable, easy to set up and capable of editing genome in non-dividing cells or cells with DNA homologous recombination deficiency.

### SUMMARY OF THE INVENTION

Disclosed herein are compositions and methods for inserting a transgene in a specific locus of a human cell. The compositions and methods of the present disclosure are useful in gene therapy and cell therapy techniques.

In one aspect, the present disclosure provides a fusion polypeptide comprising a variant of phiC31 integrase linked to a gRNA binding domain, wherein
(i) the gRNA binding domain is capable of binding to a guide RNA; and
(ii) the fusion polypeptide is capable of integrating a donor DNA sequence to locus of a genomic sequence of SEQ ID NO: 2 in a human cell.

In some embodiments, the variant of phiC31 integrase comprises the sequence of any one of SEQ ID NO: 9-13.

In some embodiments, the gRNA binding domain comprises a Cas9 protein or a fragment thereof. In some embodiments, the gRNA binding domain does not have endonuclease activity. In some embodiments, the gRNA binding domain is a dead Cas9 (dCas9). In some embodiments, the dCas9 has an amino acid sequence of SEQ ID NO: 24. In some embodiments, the gRNA binding domain is a dead Mad7 nuclease (dMad7). In some embodiments, the dMad7 has an amino acid sequence of SEQ ID NO: 34. In some embodiments, the guide RNA is capable of hybridizing a sequence in the proximity of the target genomic DNA sequence.

In some embodiments, the fusion polypeptide disclosed herein further comprises a linker that links the variant of phiC31 integrase with the gRNA binding domain. In some embodiments, the linker has the sequence of SEQ ID NO: 14-23.

In some embodiments, the fusion polypeptide disclosed herein further comprises a nuclear localization sequence (NLS). In some embodiments, the NLS has a sequence of SEQ ID NO: 35.

In another aspect, the present disclosure provides a polynucleotide encoding the fusion polypeptide disclosed herein.

In another aspect, the present disclosure provides a composition comprising (1) the fusion polypeptide disclosed herein or the polynucleotide disclosed herein, and (2) the guide RNA or the DNA encoding the guide RNA.

In another aspect, the present disclosure provides a method of integrating a transgene into the genome of a human cell. In some embodiments, the method comprises introducing into the human cell the composition disclosed herein and a donor construct comprising the transgene. In some embodiments, the human cell is a T cell or an NK cell.

These and other features, aspects, and advantages of the present invention will become better understood with regard to the following description, appended claims and accompanying drawings.

### BRIEF DESCRIPTION OF THE FIGURES

The accompanying drawings, which are incorporated herein, form part of the specification. Together with this written description, the drawings further serve to explain the principles of, and to enable a person skilled in the relevant art(s), to make and use the present invention.

FIG. 1 illustrates the sequences of target site A and a series of intermediate site A.

FIG. 2 illustrates the split-GFP site A integration efficiency assay. Wildtype phiC31 attP was placed at both site A loci in HEK293 cells. Downstream of each attP, a splice acceptor, 3′ segment of GFP, and transcription-termination sequence were also introduced. To enable detection of site-specific integration, a donor plasmid was constructed that contains the elements needed to form a complete GFP-expression cassette: CMV promoter, 5′ GFP segment, splice donor, wildtype attB site. After co-transfection of the donor and integrase-expression plasmids, cells where site-specific integration has occurred can be identified by looking for green fluorescence. Integration can happen at one (panels i and ii) or both loci (panel iii).

FIG. 3 illustrates phiC31-dCas9 (C31-dCas9) fusions for efficient localization at site A. To increase localization at site A, a series of fusions were made between WT phiC31-int and dead Cas9 (dCas9, mutant that does not cleave DNA). (FIG. 3A) We found that these fusion proteins are difficult to express, so a protein-splicing split-intein system was used to identify cells that were (1) expressing the fusion protein, and (2) were successfully transfected with the donor plasmid. In cells where both of these criteria have been met, two halves of the mCherry marker protein are trans-spliced, making the cells positive for red fluorescence. (FIG. 3B) We found that site A localization increased with higher levels of C31-int-dCas9 expression. As shown, integration efficiencies were measured at three different expression levels (mCherry-low, medium and high). (FIG. 3C) Flow cytometry plot of a representative site-A integration-efficiency experiment where cells were gated for a high level of C31-int-dCas9 expression.

FIG. 4 illustrates the results of mutant integrase activity assay. (FIG. 4A) To minimize false-positive signals, mutant integrases are subjected to a three-exon GFP plasmid inversion test. If no recombination occurs, the central GFP exon remains in the reverse orientation, which prevents production of green fluorescence above background. In cells with an active variant, the two attachment sites are recombined, which leads to inversion of exon 2 and the production of complete GFP. (FIG. 4B) Five variants with the strongest ability to recombine the attachment sites of interest were tested using the plasmid inversion assay over 96 hours in HEK293 cells (N=3, std. error). A split-intein mCherry system was used to limit analysis to cells that both received the inversion plasmid and that also robustly expressed the variant integrase (single copy expressed from H11 locus). For reference, the wildtype phiC31 integrase reaction efficiency is also shown (red). (FIG. 4C) Flow cytometry plots of representative variants and the wildtype reaction.

FIG. 5 illustrates the activity of wildtype phiC31 integrase in Jurkat cells. A three-exon GFP plasmid inversion test was performed for 96 hours in Jurkat cells to assess the ability of WT C31-int to recombine its wildtype attachment sites. RNAi was performed by electroporation of the respective DsiRNA(s) three days before electroporation of the integrase plasmids (expression and inversion-reporter vectors).

FIG. 6 illustrates a series of dead Mad7 proteins.

FIGS. 7A and 7B illustrate the expression of a phiC31-d2Mad7 fusion protein in tested HEK293 cells. (FIG. 7A) A WT phiC31 integrase gene and d2Mad7 gene were inserted at the H11 locus of a HEK293 cell. A 2A peptide gene, 5′ mCherry fragment gene and a 5′ intein fragment gene (not shown) were also inserted at the H11 locus at the downstream of the d2Mad7 gene. A donor plasmid having a CMV protomer, a 3′ intein fragment gene (not shown), 3′ mCherry fragment gene, a 2A peptide gene, a 5′ GFP fragment gene and C31 attB was then transfected to the HEK293 cell. In the cells where the C31-d2Mad7 fusion gene is expressed and a donor plasmid is transfected in the cells, two halves of the mCherry marker protein are trans-spliced via intein, making the cells positive for red fluorescence. The expression of the GFP indicates the recombination between C31 attB and attP mediated by the C31-d2Mad7 fusion protein. (FIG. 7B) The phiC31-d2Mad7 fusion protein successfully mediated the integration between C31 attB and attP located near site A.

### DETAILED DESCRIPTION OF THE INVENTION

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, the embodiments described herein can be practiced without their specific details. In other instances, methods, procedures and components have not been described in detail so as not to obscure the related relevant function being described. Also, the description is not to be considered as limiting the scope of the implementations described herein. It will be understood that descriptions and characterizations of the embodiments set forth in this disclosure are not to be considered as mutually exclusive, unless otherwise noted.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

A “coding sequence” or a sequence which “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

The “Hipp11 (H11) locus,” as used herein, refers to a “safe harbor” genomic locus that allows gene expression without disrupting internal gene function. In mice, the H11 locus is located within an intergenic region between Eif4enif1 and Drg1 genes, which are mapped close to the centromere of chromosome 11 (B Tasic et al. Proc Natl Acad Sci USA (2011) 108:7902-07). Human H11 locus is located on human chromosome 22q12.2, between the DRG1 and EIF4ENIF1 genes (F Zhu et al. Nucleic Acids Res (2014) 42:e34).

A “human cell”, as used herein, can be any cell type in a human including, for example, a cell from circulatory/immune system or organ, e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell; a cell from an endocrine system or organ, e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte); a cell from a nervous system or organ, e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph); a cell from a respiratory system or organ, e.g., a pneumocyte (a type I pneumocyte and a type II pneumocyte), a clara cell, a goblet cell, an alveolar macrophage; a cell from circular system or organ, e.g., myocardiocyte and pericyte; a cell from digestive system or organ, e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell); a cell from integumentary system or organ, e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A human cell can be normal, healthy cell; or a diseased or unhealthy cell (e.g., a cancer cell). A human cell further includes a zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell.

The term “introduce” in the context of inserting a nucleic acid sequence into a cell, means “transfection”, or ‘transformation”, or “transduction” and includes reference to the incorporation of a nucleic acid sequence into a eukaryotic or prokaryotic cell wherein the nucleic acid sequence may be present in the cell transiently or may be incorporated into the genome of the cell (e.g., chromosome, plasmid, plastid, or mitochondrial DNA), converted into an autonomous replicon. The nucleic acid sequence of the present disclosure may be introduced into a cell using any method known in the art. Various techniques for transforming animal cells may be employed, including, for example: microinjection, retrovirus mediated gene transfer, electroporation, transfection, or the like (see, e.g., Keown et al., Methods in Enzymology 1990, 185:527-537).

As used herein, “locus” refers to a specific location on a chromosome. A known locus can contain known genetic information, such as one or more polymorphic marker sites.

The terms “polynucleotide” and “nucleic acid sequence” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

As used herein, the term “vector” refers to a nucleic acid molecule capable of transporting between different genetic environments another nucleic acid to which it has been operatively linked. Preferred vectors are those capable of autonomous replication and expression of structural gene products present in the DNA segments to which they are operatively linked. Vectors, therefore, preferably contain the replicons and selectable markers described earlier. Vectors include, but are not necessarily limited to, expression vectors.

The term “transgene” refers to an exogenous polynucleotide introduced into a host cell (e.g., a HEK293 cell), irrespective of the method used for the introduction. The methods include those known in the art, including vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides).

The term “variant,” when used in conjunction with a gene, a nucleotide sequence or a protein, refers to a gene, a nucleotide sequence or protein that is different from the reference or original gene, nucleotide sequence or protein in at least one nucleotide or amino acid residue. In certain circumstances, the term “variant” is used interchangeably with the term “mutant.”

As used herein, the term “vector” refers to a polynucleotide molecule that comprises a gene or a nucleic acid sequence of particular interest. Typically, the construct also includes appropriate regulatory sequences. For example, the polynucleotide molecule can include regulatory sequences located in the 5′-flanking region of the nucleotide sequence encoding the guide RNA and/or the nucleotide sequence encoding a site-directed modifying polypeptide, operably linked to the coding sequences in a manner capable of expressing the desired transcript/gene in a host cell.

### Fusion Protein for Inserting Transgene

In one aspect, the present disclosure provides a fusion polypeptide capable of integrating a donor DNA sequence to a locus containing the sequence of SEQ ID NO: 2 in a human cell. In some embodiments, the fusion protein comprises a variant of phiC31 integrase linked to a gRNA binding domain, wherein the gRNA binding domain is capable of binding to a guide RNA. In some embodiments, the guide RNA is capable of hybridizing to a DNA sequence in the proximity of the target genomic sequence of SEQ ID NO: 2.

phiC31 Integrase

PhiC31 integrase is a site-directed recombinase derived from bacteria phage phiC31. Site-specific recombinase refers to a family of highly specialized enzymes that promote DNA rearrangement between specific target sites (Greindley et al., 2006; Esposito, D., and Scocca, J. J., Nucleic Acids Research 25, 3605-3614 (1997); Nunes-Duby, S. E., et al, Nucleic Acids Research 26, 391-406 (1998); Stark, W. M., et al, Trends in Genetics 8, 432-439 (1992)). Virtually all site-specific recombinases can be categorized within one of two structurally and mechanistically distinct groups: the tyrosine (e.g., Cre, Flp, and the lambda integrase) or serine (e.g., phiC31 integrase, Bxb1 integrase, gamma-delta resolvase, Tn3 resolvase and Gin invertase) recombinases. Both recombinase families recognize target sites composed of two inversely repeated binding elements that flank a spacer sequence where DNA breakage and religation occur. The recombination process requires concomitant binding of two recombinase monomers to each target site: two DNA-bound dimers (a tetramer) then join to form a synaptic complex, leading to crossover and strand exchange. In particular, recombinases can recognize endogenous sequences in a genome of interest.

Integrases, or uni-directional recombinase, refer to recombinase enzymes whose recognition sites are destroyed after the recombination has taken place. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Binding sites for uni-directional recombinases, such as phiC31 and Bxb1 integrase, are traditionally called attB and attP (i.e., the target sites of the integrase). These sites have a minimal length of approximately 34-40 base pairs (bp) (Groth A C et al., Proc. Natl. Acad. Sci. USA 97, 5995-6000 (2000)). These sites are typically arranged as follows: AttB comprises a first DNA sequence attB5′, a core region, and a second DNA sequence attB3′ in the relative order attB5′-core region-attB3′. AttP contains a first DNA sequence (attP5′), a core region, and a second DNA sequence (attP3′) in the relative order attP5′-core region-attP3′. The recombinase mediates production of recombination-product sites that can no longer act as substrates for the recombinase. The recombination-product sites contain, for example, the relative order attL5′-recombination-product site-attR3′, in which attL is hybrid sequence of attB5′ and attP3′, whereas attR is hybrid sequence of attB3′ and attP5′. In some cases, the sites can be variants of the native attP/attB sequences, such as tandem repeats (e.g., three repeats such as attPx3), truncated sequences, or both. In some embodiments, the first recombination site and the second recombination site are attP and attB, respectively, or vice versa.

Guide RNA (gRNA) Binding Domain

As used herein, a gRNA binding domain refers to a polypeptide sequence that contains a Cas protein or fragment thereof, which is capable of binding to a guide RNA and directs a protein containing the gRNA binding domain to a nucleic acid sequence targeted by the guide RNA.

In general, a “guide RNA” refers to an RNA that directs sequence-specific binding of a protein complex to the target sequence. Typically, a guide RNA comprises (i) a guide sequence that has sufficient complementarity with a target polynucleotide sequence to hybridize with the target sequence and (ii) a trans-activating cr (tracr) mate sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length.

In the context of formation of a protein complex, a “target sequence” or “a sequence of a target DNA” refers to a sequence to which a guide sequence is designed to have complementarity, where hybridization between a target sequence and a guide sequence promotes the formation of a protein complex. Full complementarity is not necessarily required, provided there is sufficient complementarity to cause hybridization and promote formation of a protein complex. A target sequence may comprise any polynucleotide, such as DNA or RNA polynucleotides or DNA/RNA hybrid polynucleotides. In some embodiments, a target sequence is located in the nucleus or cytoplasm of a cell.

In some embodiments, the guide RNA comprises a guide sequence fused to a tracr sequence, i.e., the tracr sequence and tracr mate sequence are contained within a single transcript, such that hybridization between the two produces a transcript having a secondary structure, such as a hairpin. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the present application, the guide RNA has at least two or more hairpins. In preferred embodiments, the guide RNA has two, three, four or five hairpins. In a further embodiment of the invention, the guide RNA has at most five hairpins. In some embodiments, the guide RNA further includes a transcription termination sequence, preferably a polyT sequence, for example six T nucleotides. In some embodiments, the tracr sequence is a separate transcript from a transcript comprising the tracr mate sequence.

In some embodiments, the gRNA binding domain comprises a Cas protein or a fragment of a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx 3, Csx1, Csx15, Csf1, Csf2, Csf3, Csf4, homologs thereof, or modified versions thereof.

In some embodiments, the Cas protein is mutated such that the mutated Cas protein lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, the fragment of the Cas protein lacks DNA cleavage activity, e.g., the fragment does not contain the catalytic domain of the Cas protein (e.g., RuvC I, RuvC II, and RuvC III domain of Cas9). In some embodiments, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a Cas protein is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other Cas protein is from a species other than S. pyogenes, mutations in corresponding amino acids may be made to achieve similar effects.

In some embodiments, the gRNA binding domain comprises a Mad7 nuclease (ErCas12a, SEQ ID NO: 33) or a fragment thereof. Mad7 only requires a crRNA for gene editing and allows for specific targeting of AT rich regions of the genome. Mad7 cleaves DNA with a staggering cut as compared to SpCas9 which has blunt cutting. In some embodiments, the gRNA binding domain comprises a dead Mad7 substantially lacking all DNA cleavage activity. In some embodiments, the mutated residues of dMad7 include D877, E962, and D1213. In some embodiments, the mutation of dMad7 include D877A, E962A, and D1213A, or a combination thereof.

### Linker

In some embodiments, the fusion polypeptide disclosed herein further comprises a linker that links the variant of phiC31 integrase with the gRNA binding domain. In some embodiments, the linker comprises a glycine-serine (GS) doublet between 2 and 20 amino acid residues in length. Exemplary GS doublets include (G₄S)₃. In some embodiments, the linker has the sequence of SEQ ID NO: 14-23.

Methods of Screening a Variant phiC31 Integrase

In another aspect, the present disclosure provides a method of screening a fusion polypeptide comprising a variant phiC31 linked to a gRNA binding domain wherein the fusion polypeptide is capable of integrating a donor DNA sequence to a sequence of SEQ ID NO: 2 in a human cell genome.

### Cell Line Containing Recombination Site

In one embodiment, the screen method starts with obtaining a cell line which comprises at a genomic locus a uni-directional recombination site recognized by a unidirectional recombinase other than phiC31 (e.g., Bxb1). In some embodiments, the genomic locus containing the recombination site is a region that provides increased expression of transgene contained in the region. Examples of such locus include without limitation, ROSA26, ROSA26 like locus, HPRT, AAVS1 and Hipp11 (H11). In a preferred embodiment, the locus is H11.

The method of generating a cell line containing the recombination site of interest in a target locus is known in the art. See, e.g., Duportet X et al., “A platform for rapid prototyping of synthetic gene networks in mammalian cells.” Nucleic Acids Res. (2014) 1; 42(21):13440-51; and Matreyek K et al., “A platform for functional assessment of large variant libraries in mammalian cells” Nucleic Acids Res (2017) 45(11):e102.

Typically, a nucleic acid construct comprising the recombination site of interest flanked by homology arms of the target locus is created. The nucleic acid construct may also include additional nucleic acid fragments that facilitate the generation of the cell line, e.g., selection marker sequences. In one embodiment, the nucleic acid construct contains a hygromycin resistance marker.

In some embodiments, the nucleic acid construct may also include additional nucleic acid fragments that facilitate selection of variants of a target gene, such as promoter sequences, which will be inserted to the target locus together with the recombination site. In some embodiments, the nucleic acid construct comprises a tetracycline (Tet) responsive promoter and an EF-1 promoter.

When the nucleic acid construct containing the recombination site of interest is introduced into a cell, the recombination site can be inserted into the target locus through homologous recombination. In certain embodiments, a site-specific nuclease is expressed in the cell to generate a double strand break in order to increase the efficiency of homologous recombination. In some embodiments, the site-specific nuclease is a CRISPR/Cas protein, a zinc finger nuclease (ZFN) or a transcriptional activator-like effector nuclease (TALEN).

### Variant Library

In some embodiments, the screen method further comprises generating a cell library using the cell line that comprises the unidirectional recombination site and a library of nucleic acid constructs, each of the nucleic acid constructs comprising a second unidirectional recombination site recognized by the unidirectional recombinase, and a variant of phiC31 integrase gene.

Methods of generating variants of a target gene is known in the art. See, e.g., Zhou Y H et al., “Random mutagenesis of gene-sized DNA molecules by use of PCR with Taq DNA polymerase.” Nucleic Acids Res. (1991)19(21):6052; Engler C et al., “Golden Gate Shuffling: A One-Pot DNA Shuffling Method Based on Type IIs Restriction Enzymes” PLoS One. 2009; 4(5):e5553; and Ashraf M et al., “ProxiMAX randomization: a new technology for non-degenerate saturation mutagenesis of contiguous codons.” Biochem Soc Trans. (2013) 41(5):1189-94.

Zhou Y H et al has reported a simple method of random mutagenesis using Taq DNA polymerase, which lacks a 3′-5′ exonucleolytic editing activity and thus becomes error-prone (Nucleic Acids Res. (1991)19(21):6052). Engler C et al developed a protocol to assemble multiple DNA fragments together into a vector, allowing the generation of libraries of recombinant genes by combining several fragment sets prepared from different parental templates (PLoS One. 2009; 4(5):e5553). The protocol can shuffle the DNA fragments derived from templates having no homology and can be used to introduce any variation in any part of a given gene.

Ashraf M et al developed a randomization method of generating DNA cassettes for saturation mutagenesis, i.e., replacing of wild-type codons with codons for all 20 amino acids, without degeneracy or bias (Biochem Soc Trans. (2013) 41(5):1189-94, which is incorporated herein by reference). In short, double-stranded DNA donors, carrying randomized codon at their termini, are ligated individually on to a double-stranded DNA acceptor sequence, which is phosphorylated at the 5′ end only. After ligation, the products are amplified, purified, quantified and then combined in the required ratios. The combined product is digested with MlyI, which generates a double-stranded DNA consisting of the acceptor sequence plus the randomized codon at the 5′ end. The process is then repeated, using the double-stranded DNA product from the previous cycle as the acceptor for the next round of ligation. As a result, saturation mutagenesis can be introduced to contiguous codons.

The combination of the above methods and alike can generate any and all variants of phiC31 integrase gene.

The variants of phiC31 integrase gene can be cloned to a nucleic acid vector to generate a library of nucleic acid constructs that includes all variants of phiC31 integrase gene. Suitable eukaryotic vectors from which one can construct the nucleic acid constructs are well known in the art. See, for example, Broach, Cell (1982) 28:203-204; Dilon et al., J. Clin. Hematol. Oncol. (1980) 10:39-48; Maniatis, In: Cell Biology: A Comprehensive Treatise, Vol. 3, Gene Sequence Expression, Academic Press, NY, pp. 563-608, 1980.

The library of nucleic acid constructs is then introduced to the cell line to generate a cell library. The nucleic acid constructs can be introduced to the cell line using methods known in the art, such as transformation or transfection. The recombinase that recognizes the recombination sites are expressed in the cell line, which mediates the recombination between the first and the second recombination sites, resulting in the incorporation of the variants of the target gene to the target genomic locus of the cell line. In some preferred embodiments, the concentration of the library of nucleic acid constructs is adjusted so that single variant is introduced into each cell. In some embodiments, the cell library can be enriched with a selection marker.

### Selecting Desired Variant

A screening method can be designed to select desired variant of phiC31 integrase gene from the library of cells.

In one embodiment, a screen method can be designed to select a phiC31 integrase variant that recognizes a variant of recombination site (i.e., a mutant recombination site), e.g., a pseudo-recombination site. In such a screen method, a selection construct is generated to comprises: a third unidirectional recombination site, a third promoter, a fourth unidirectional recombination site, and a selectable marker (e.g., an antibiotic resistance gene), wherein at least one of the third and the fourth unidirectional recombination sites is a variant or mutant that is not recognized by wildtype phiC31 integrase but recognized by the desired variant of phiC31 integrase, and wherein the third promoter and the selectable marker is arranged in opposite orientation.

The selection construct is then introduced to the cell library that comprises the variants of phiC31 integrase gene. The transformed cell library is maintained under conditions that facilitate recombination between the third and the fourth unidirectional recombination sites mediated by the desired variant of phiC31 integrase, thereby reversing the orientation of the third promoter or the selectable marker in the selection construct. The third promoter (or the selectable marker) after reversing the orientation then direct the expression of the selectable marker (or be directed by the third promoter when the selectable marker reverses the orientation). Therefore, when the transformed cell library is subject to the selectable condition, e.g., in the presence of antibiotics, the cell containing the desired variant of the integrase can be selected.

Theoretically, the method disclosed herein can generate any and all desired phiC31 integrase variant by using a sufficiently large variant library. In practice, however, due to the size limit of the variant library, a desired variant may not be found in one round of selection if it is different from the original gene/protein in too many positions, e.g., nucleotide residues or amino acid residues. Therefore, in certain embodiments, the selecting method disclosed herein involves stepwise selection of desired variant by generating a series of intermediate variants, each intermediate variant is different in just a few positions from the original gene or the intermediate variant gene generated in the previous round of selection.

In one exemplary embodiment, the method disclosed herein selects a variant phiC31 integrase that can recognize site A (SEQ ID NO: 2). The sequence of site A is different from the wildtype attP site in more than 50% of the nucleotide residues, e.g., 26 out of 48 nucleotide residues are different. To obtain a variant integrase that recognizes the site A, an intermediate mutant attP sequences, e.g., a sequence selected from SEQ ID NOs: 3-7, is created to identify an intermediate variant phiC31(varC31) integrase that recognizes the intermediate mutant attP sequence from a mutant integrase library. The identified intermediate variant integrase gene is then used as the start integrase gene to generate a mutant integrase library, which is used in the next round of selection to identify variant integrase that recognizes the site A or a second intermediate variant integrase that recognizes a second intermediate mutant attP sequence more similar to site A as compared to the intermediate mutant attP sequence used in the previous round of selection.

### Methods of Inserting Transgene

The present disclosure in another aspect provides a method of inserting a transgene into the locus containing the sequence of SEQ ID NO: 2 in a human cell. In some embodiments, the method comprises introducing into the human cell the composition disclosed herein (e.g., a composition comprising the fusion protein disclosed herein or a nucleic acid encoding the fusion disclosed herein) and a donor construct comprising the transgene.

In some embodiments, the method of inserting a transgene into the locus containing the sequence of SEQ ID NO: 2 as disclosed herein comprises introducing into the human cell one or more vectors comprising (1) the nucleic acid encoding the varC31-Cas fusion protein disclosed herein, (2) a guide RNA targeting a genomic sequence in the vicinity of varC31 targeting sequence of SEQ ID NO: 2 or a nucleic acid encoding such guide RNA; and (3) the transgene. In some embodiments, the varC31-Cas fusion protein and guide RNA can be introduced into the human cell as a protein/RNA form, so called RNP form.

In some embodiments, the one or more vectors are introduced into the human cell via conventional non-viral or viral based gene transfer methods. Non-viral vector delivery systems include DNA plasmids, RNA (e.g. a transcript of a vector described herein), naked nucleic acid, and nucleic acid complexed with a delivery vehicle, such as a liposome, protein complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, electroporation, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (in vivo). Conventional viral based systems could include retroviral, lentiviral, adenoviral, adeno-associated and herpes simplex viral vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include HEK293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a cell transfected with one or more vectors described herein is used to establish a new cell line comprising one or more vector-derived sequences. In some embodiments, a cell transiently transfected with the components of the composition as described herein (such as by transient transfection of one or more vectors, or transfection with RNA, or transfection with protein), and modified through the activity of the complex, is used to establish a new cell line comprising cells containing the modification but lacking any other exogenous sequence. In some embodiments, cells transiently or non-transiently transfected with one or more vectors described herein, or cell lines derived from such cells are used for gene therapy or cell therapy.

The foregoing description of the invention has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. It is intended that the appended claims be construed to include other alternative embodiments of the invention; including equivalent components, methods, and means.

### Example 1

This example illustrates the selection of a candidate DNA sequence in human genome that is likely to be recognized by a variant phiC31 integrase to mediate recombination at the locus containing the candidate DNA sequence.

The selection was started with searching the human genome for the sites that are similar to the wild type attP sequence recognized by wildtype phiC31 integrase and also meet the following requirements: (1) the site has a medium GC content (20-60%); (2) the site has a unique sequence in human genome; (2) the site is intergenic (i.e., between gene coding regions); and (3) the site is not next to oncogene or anti-oncogene. The expression of the gene neighboring to the site and whether the site is proximate to hypersensitive site (HSS) were also assessed to determine whether the site should be selected. In the initial screening, approximately one million sites met the requirements.

About 75 sites with highest similarity to the wildtype attP sequence were then manually inspected to narrow the list down to six sites, namely, sites A, B, C, D, E and F. Ultimately, site A (SEQ ID NO: 2) was selected to screen phiC31 variants.

Site A is located on human chromosome 4, short arm, band 14 region. The most recent gene at the 5′ of site A is about 65 kB away and named “LINC01258”, which encodes a non-coding RNA that targets gene PCGF5. PCGF5 plays key role in differentiation and the NOTCH signaling pathway, and the expression of LINC01258 is up-regulated in type I diabetes (Zhang Z et al. Comparative analysis of the DNA methylation landscape in CD4, CD8, and B memory lineages. Clin Epigenetics. 2022 Dec. 15; 14(1):173).

The closest gene at the 3′ of site A is about 20 kB away and named “KLF3-AS1”, which activates KLF3 (an anti-oncogene). KLF3-AS1 was reported to promotes cartilage repair (Liu Y et al. Exosomal KLF3-AS1 from hMSCs promoted cartilage repair and chondrocyte proliferation in osteoarthritis. Biochem J. 2018 Nov. 28; 475(22):3629-3638).

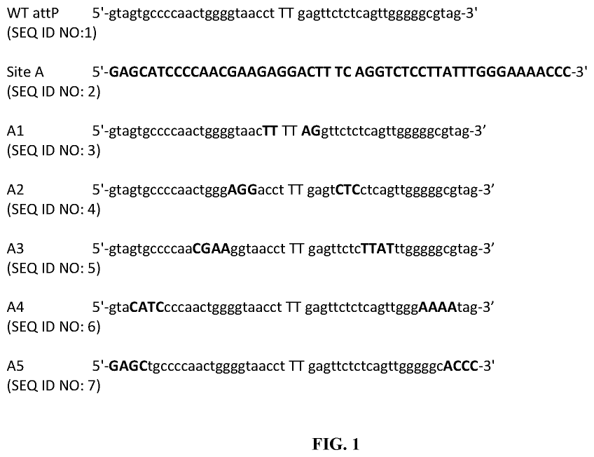

The comparison between site A and phiC31 attP sequence is shown in FIG. 1. The 2-base cross-over cores are not identical between site A and phiC31 attP sequence (TT vs. TC). However, integrase does not check these bases. Between site A and phiC31 attP sequence, 22 of 48 (46%) bases in half-sites are identical, while 38 of 48 (79%) bases in half-sites are similar. The symmetry of site A and phiC31 attP sequence was measured by the comparison of one half-site to the complementary sequence of the other half-site. In site A, 15 of 24 bases are identical and 21 of 24 bases are similar. In phiC31 attP sequence, 15 of 24 bases are identical and 19 of 24 bases are similar.

### Example 2

This example demonstrates the generation of a phiC31-dCas9 fusion protein that has increased efficiency of locating to site A with the help of a gRNA.

Based on serine integrase structures published to date, the regions of the integrase polypeptide responsible for DNA interaction are likely to be far away from those that have an impact on localization (Rutherford et al. Attachment site recognition and regulation of directionality by the serine integrases. Nucleic Acids Res. 2013, 41(17):8341-8356). As a result, while our screening method generally limits the mutations to the regions of phiC31 integrase that impact integration specificity and efficiency, the ability of our variants to reach site A is unlikely to be changed from that of the wildtype (WT) phiC31 integrase. To maximize the efficiency that an integrase variant localizes to site A and thus increasing the integration efficiency, we tried to optimize the localization of wildtype phiC31 integrase (WT C31-int) at site A.

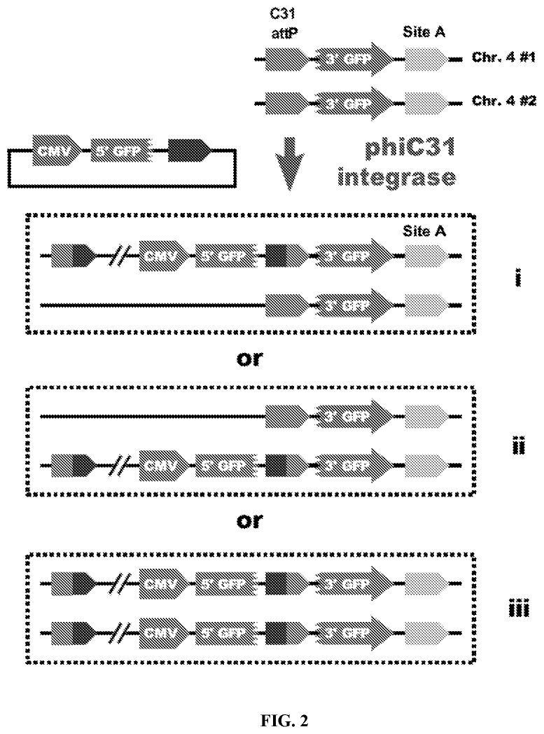

To estimate WT C31-int integration efficiency at site A, we knocked-in a split-GFP recombination reporter at both loci of site A (FIG. 2) via Cas9-stimulated non-homologous end-joining. As illustrated in FIG. 2, wildtype phiC31 attP was placed at both site A loci in HEK293 cells. Downstream of each attP, a splice acceptor, 3′ segment of GFP, and transcription-termination sequence were also introduced. To enable detection of site-specific integration, a donor plasmid was constructed that contains the elements needed to form a complete GFP-expression cassette: CMV promoter, 5′ GFP segment, splice donor, wildtype attB site. After co-transfection of the donor and integrase-expression plasmids, cells where site-specific integration has occurred can be identified by looking for green fluorescence. Integration can happen at one (panels i and ii) or both loci (panel iii). This system allows for measurement of integration efficiency at site A within 2-4 days, and thus enables a more precise calculation of localization efficiency, which we define as the ability of C31-int to reach site A under the tested experimental conditions (Table 1).

To improve the localization of C31-int at site A, we have tested the co-expression of various proteins that may improve general expression or site A accessibility, including SV40 Large-T (SV40 LT) and C31-int-dCas9 fusions (Table 1). dCas9 fusions have proven to be the most potent way to improve localization at site A, although they are difficult to express.

TABLE 1
Site A localization efficiency measurement.

| | | | dCas9 | dCas9 | dCas9 |
|---|---|---|---|---|---|
| Expressed | | Integrase + | fusion, | fusion, | fusion, high |
| Protein | Integrase | SV40 LT | low expr. | med expr. | expr. |
| Localization | 14.5% | 19.1% | 32.3% | 40.6% | 49.5% |
| efficiency | | | | | |

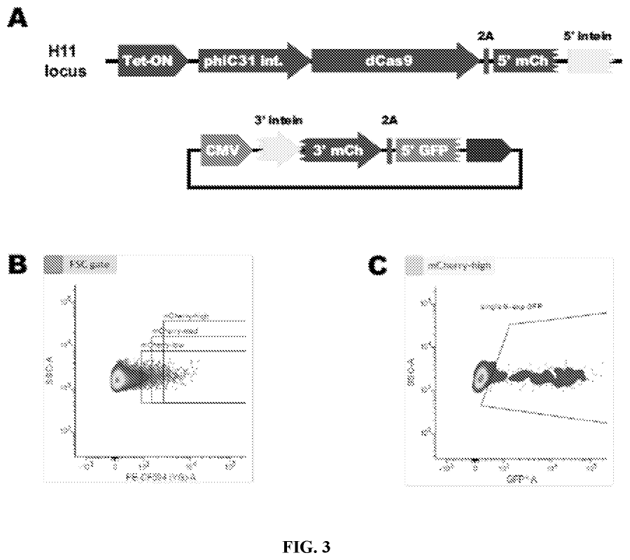

The expression of a phiC31-dCas9 (C31-dCas9) fusion protein in tested HEK293 cells (in which wildtype phiC31 attP and 3′ segment of GFP was placed at site A loci) is illustrated in FIG. 3A-3C. To increase localization at site A, a series of fusions were made between WT phiC31-int and dead Cas9 (dCas9, mutant that does not cleave DNA) and inserted at the H11 locus (FIG. 3A). We found that these fusion proteins are difficult to express, so a protein-splicing split-intein system (FIG. 3A) was used to identify cells that were (1) expressing the fusion protein, and (2) were successfully transfected with the donor plasmid. In the system, a 5′ mCherry fragment gene and a 5′ intein fragment gene is inserted at the downstream of the C31-dCas9 fusion gene in the H11 locus. In the cells where both of these criteria have been met, i.e., the C31-dCas9 fusion gene is expressed and a donor plasmid is transfected in the cells, two halves of the mCherry marker protein are trans-spliced via intein, making the cells positive for red fluorescence. As shown in FIGS. 3B and 3C, integration efficiencies were measured at three different expression levels (mCherry-low, medium and high) and site A localization increased with higher levels of C31-int-dCas9 expression.

### Example 3

This example demonstrates the directed evolution approach that was used to screen phiC31 integrase variants recognizing site A.

### Generating a Mammalian Cell Library Expressing Variants

In the first step, we generated a mammalian cell library platform that expresses a population of gene variants wherein each cell contains a defined genetic alteration. Such mammalian cell library platform has been disclosed in U.S. patent Ser. No. 11/492,613. In short, a landing pad that contains a Bxb attP site was first inserted into the genome of a mammalian cell line. The existence of the Bxb attP site in the genome allows the mammalian cell line to receive a group of heterologous genes (i.e., gene variants) in later steps via Bxb integrase-mediated integration, thus generating a cell library wherein each cell contains one specific gene variant.

To begin with, an HEK293 cell line was created to contain a landing pad to receive heterologous genes at intergenic Hipp1 (H11) locus e.g., via TALE nuclease-stimulated homologous recombination. The landing pad comprises a Bxb1 attP site flanked by a Tet promoter and an EF-1 promoter.

A library of nucleic acid constructs containing mutant phiC31 integrase genes was then generated using repeated error prone PCR, and single or multi-codon saturation. Each of the nucleic acid construct in the mutant phiC31 library contains a Bxb1 attB site, a blasticidin resistance marker and a mutant phiC31 integrase.

The library of nucleic acid constructs described above was then introduced into the HEK293 cell line above together with an optimized Bxb1 integrase expression construct to generate a cell library. Upon the expression of Bxb1 integrase, the recombination between the Bxb1 attB and attP sites resulted in the incorporation of a mutant phiC31 integrase in the H11 locus under the control of the Tet promoter and the blasticidin resistance gene under the control of the EF-1 promoter.

### Directed Evolution Approach

Considering that it may be difficult to directly screen a phiC31 integrase variant that recognizes Site A in one round of screening, we generated a series of intermediate Site A sequences (SEQ ID NOS: 3-7) for screening intermediate phiC31 integrase variants. As shown in FIG. 1, we broke site A into 5 segments with 3-7 base changes per segment compared to wildtype phiC31 attP. The segments were grouped into regions based on predicted proximity to key amino acids in phiC31 integrase.

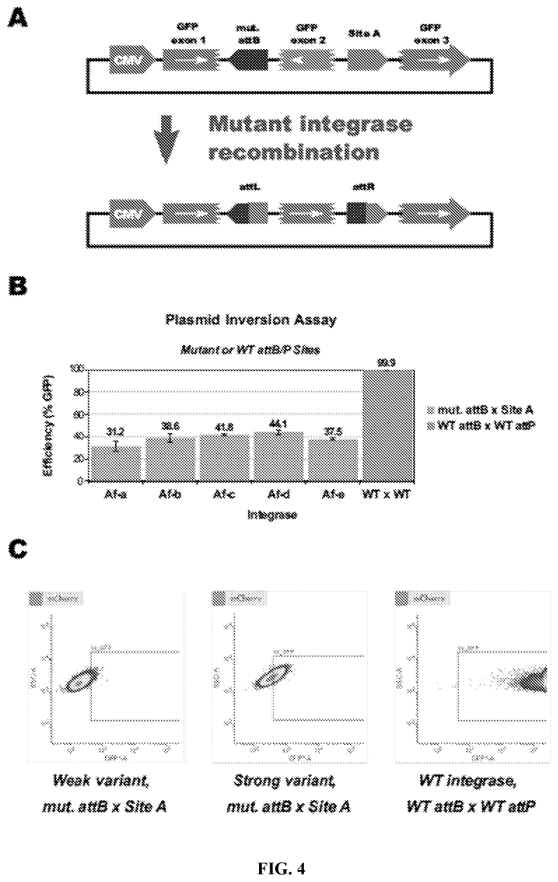

Using the intermediate substrates, we isolated variants from saturation libraries that recognize small att-site intermediates. We then combined these mutants to create integrase variants that are active on the full site A sequence in a plasmid inversion assay (FIG. 4). As shown in FIG. 4A, to minimize false-positive signals, mutant integrases were subjected to a three-exon GFP plasmid inversion test. If no recombination occurs, the central GFP exon remains in the reverse orientation, which prevents production of green fluorescence above background. In cells with an active variant, the two attachment sites are recombined, which leads to inversion of exon 2 and the production of complete GFP. As shown in FIG. 4B, five variants with the highest efficiency to recombine the attachment sites of interest were tested using the plasmid inversion assay over 96 hours in HEK293 cells (N=3, std. error). A split-intein mCherry system (see Example 3 and FIG. 3A) was used to limit analysis to cells that both received the inversion plasmid and that also robustly expressed the variant integrase (single copy expressed from H11 locus). For reference, the wildtype phiC31 integrase reaction efficiency is also shown (red). FIG. 4C shows the flow cytometry plots of representative variants and the wildtype reaction.

While we have observed promising ranges of activity from these variants (31-44%, FIG. 4B), the level of recombination per cell is still majorly reduced relative to the wildtype reaction (FIG. 4B). It's possible that this is a false-negative consequence of a slower reaction rate, as a higher level of double-stranded RNA would be present (spanning GFP exon 2) if the reaction proceeds more slowly, leading to RNA interference against GFP. We test our most active variants for integration at site A, the efficiencies fall in the ~5-22% range (Table 2).

TABLE 2
Projected site A integration efficiencies in HEK293 cells.

| | | | dCas9 | dCas9 | dCas9 |
|---|---|---|---|---|---|
| Expressed | | Integrase + | fusion, | fusion, | fusion, high |
| Protein | Integrase | SV40 LT | low expr. | med expr. | expr. |
| Observed | 15% | 19% | 32% | 41% | 50% |
| Localization | | | | | |
| Observed | 31-44% | 31-44% | 31-44% | 31-44% | 31-44% |
| variant | | | | | |
| inversion | | | | | |
| efficiency | | | | | |
| Projected | 5-7% | 6-8% | 10-14% | 13-18% | 16-22% |
| site A | | | | | |
| integration | | | | | |
| efficiency | | | | | |

### Example 4

This example illustrates integration activity of wildtype phiC31 integrase in human T lymphocyte cell line Jurkat cells.

In parallel to the HEK293 work described above, we started experiments in Jurkat cells to prepare for site A integration of therapeutic donor plasmids in primary T and HSPCs. As previously described by Maucksch et al. (Cell type differences in activity of the Streptomyces bacteriophage phiC31 integrase. Nucleic Acids Res. 2008, 36(17):5462-71), we found that C31-int activity in Jurkat cells is majorly inhibited (FIG. 5). Maucksch et al. observed that DAXX, a known inhibitor of C31-int, is strongly expressed in Jurkat, so we tested RNAi knock-down of it and two other known C31-int inhibitors (Sp100, TTRAP; FIG. 4). Knockdown of DAXX alone was enough to double C31-int activity in Jurkat (from 16.7% to 34.1%, N=2). However, there are still clearly additional inhibitors present.

### Example 5

This example demonstrates the generation of a phiC31-dMad7 fusion protein that has increased efficiency of locating to site A with the help of a gRNA.

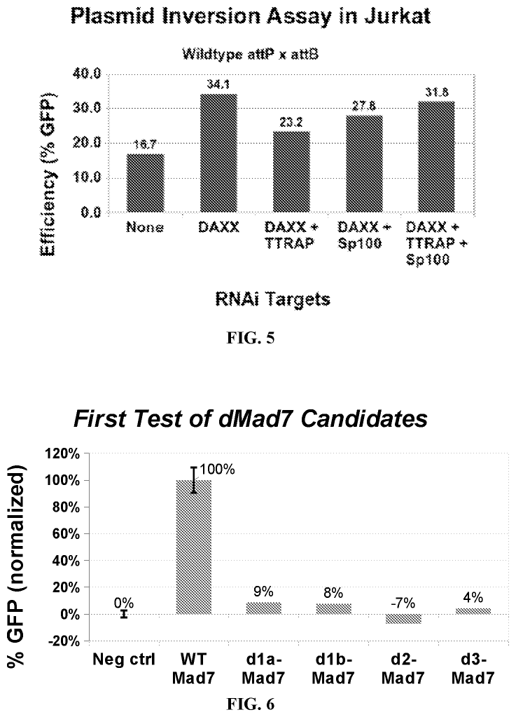

We first generated a dead Mad7 protein that lacks cleavage activity. It has been reported that the catalytic residues in Mad7 are D877, E962 and D1213. We generated a series of dMad7 candidates, including d1aMad7 (D877A), d1bMad7 (E962A), d2Mad7 (D877A, E962A), and d3Mad7 (D877A, E962A, D1213A). We found that d2Mad7 has the lowest cleavage activity (FIG. 6).

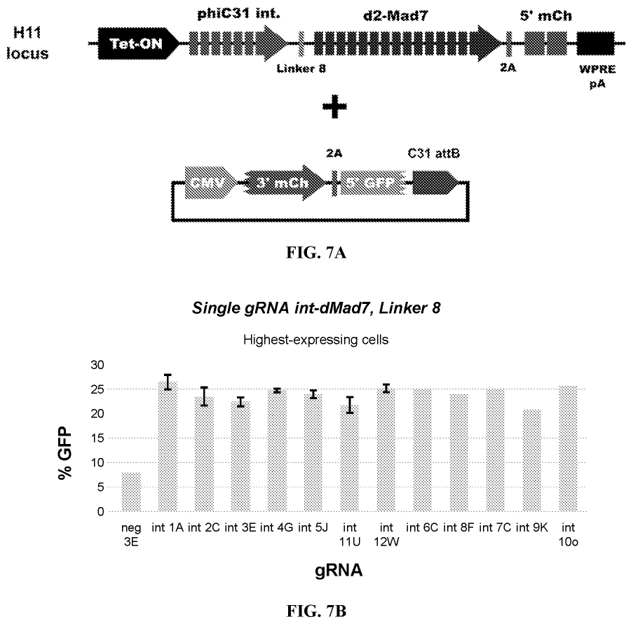

The expression of a phiC31-d2Mad7 fusion protein in tested HEK293 cells is similar to the expression of a phiC31-dCas9 fusion protein of Example 2 and is illustrated in FIGS. 7A and 7B. A WT phiC31 integrase gene and d2Mad7 gene were inserted at the H11 locus (FIG. 7A) of a HEK293 cell which contains wildtype phiC31 attP and 3′ segment of GFP at site A loci (see FIG. 2). A 2A peptide gene, 5′ mCherry fragment gene and a 5′ intein fragment gene (not shown) were also inserted at the H11 locus at the downstream of the d2Mad7 gene (FIG. 7A). A donor plasmid having a CMV protomer, a 3′ intein fragment gene (not shown), 3′ mCherry fragment gene, a 2A peptide gene, a 5′ GFP fragment gene and C31 attB was then transfected to the HEK293 cell. In the cells where the C31-d2Mad7 fusion gene is expressed and a donor plasmid is transfected in the cells, two halves of the mCherry marker protein are trans-spliced via intein, making the cells positive for red fluorescence. The expression of the GFP indicates the recombination between C31 attB and attP mediated by the C31-d2Mad7 fusion protein (see FIG. 2). As the recombination mediated by C31 requires a tetramer, we also expressed WT phiC31 integrase in the cells to increase the recombination efficiency in some experiments. As shown in FIG. 7B, the phiC31-d2Mad7 fusion protein successfully mediated the integration between C31 attB and attP located near site A.

What is claimed is:

1. A fusion polypeptide comprising a variant of phiC31 integrase linked to a gRNA binding domain, wherein
(i) the gRNA binding domain is capable of binding to a guide RNA; and
(ii) the fusion polypeptide is capable of integrating a donor DNA sequence to locus of a genomic sequence of SEQ ID NO: 2 in a human cell.

2. The polypeptide of claim 1, wherein the variant of phiC31 integrase comprises the sequence of any one of SEQ ID NO: 9-13.

3. The polypeptide of claim 1, wherein the gRNA binding domain comprises a Cas9 protein or a fragment thereof.

4. The polypeptide of claim 1, wherein the gRNA binding domain does not have endonuclease activity.

5. The polypeptide of claim 1, wherein the gRNA binding domain is a dead Cas9 (dCas9).

6. The polypeptide of claim 1, wherein the gRNA binding domain comprises an amino acid sequence of SEQ ID NO: 25.

7. The polypeptide of claim 1, wherein the gRNA binding domain is a dead Mad7 (dMad7).

8. The polypeptide of claim 1, wherein the gRNA binding domain comprises an amino acid sequence of SEQ ID NO: 34.

9. The polypeptide of claim 1, wherein the guide RNA is capable of hybridizing to a sequence in the proximity of a genomic sequence of SEQ ID NO: 2.

10. The polypeptide of claim 1, further comprising a linker that links the variant of phiC31 integrase with the gRNA binding domain.

11. The polypeptide of claim 10, wherein the linker has the sequence of any one of SEQ ID NO: 14-23.

12. The polypeptide of claim 1, further comprising a nuclear localization sequence (NLS).

13. The polypeptide of claim 12, wherein the NLS has a sequence of SEQ ID NO: 35.

14. A polynucleotide encoding the fusion polypeptide of any one of claims 1-13.

15. A composition comprising (1) the fusion polypeptide of any one of claims 1-13 or the polynucleotide of claim 14, and (2) the guide RNA or the DNA encoding the guide RNA.

16. A method of integrating a transgene into the genome of a human cell comprising introducing into the human cell the composition of claim 15 and a donor construct comprising the transgene.

17. The method of claim 16, wherein the human cell is an iPSC, T cell or an NK cell.


SEQUENCE LISTING (35 sequences)

SEQ ID NO: 1 - DNA, 50 bp, Lomovskayavirus C31
  source 1..50: mol_type = genomic DNA
gtagtgccccaactggggtaacctttgagttctctcagttgggggcgtag

SEQ ID NO: 2 - DNA, 50 bp, Homo sapiens
  source 1..50: mol_type = genomic DNA
gagcatccccaacgaagaggactttcaggtctccttatttgggaaaaccc

SEQ ID NO: 3 - DNA, 50 bp, synthetic construct
  source 1..50: mol_type = other DNA
gtagtgccccaactggggtaacttttaggttctctcagttgggggcgtag

SEQ ID NO: 4 - DNA, 50 bp, synthetic construct
  source 1..50: mol_type = other DNA
gtagtgccccaactgggaggacctttgagtctcctcagttgggggcgtag

SEQ ID NO: 5 - DNA, 50 bp, synthetic construct
  source 1..50: mol_type = other DNA
gtagtgccccaacgaaggtaacctttgagttctcttatttgggggcgtag

SEQ ID NO: 6 - DNA, 50 bp, synthetic construct
  source 1..50: mol_type = other DNA
gtacatccccaactggggtaacctttgagttctctcagttgggaaaatag

SEQ ID NO: 7 - DNA, 50 bp, synthetic construct
  source 1..50: mol_type = other DNA
gagctgccccaactggggtaacctttgagttctctcagttgggggcaccc

SEQ ID NO: 8 - protein, 605 aa, Lomovskayavirus C31
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 9 - protein, 605 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRWLGGYVGGKAKKGFEYVSETKEITRNGRMVNVVINKLAHSTTPLTGPFEFEPDVIRWWWREIKTHKHLPFKPGSQAAIHPGSITGLCKRMDADAVPTRGETIGKKTASSAWDTGTVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGSYVIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 10 - protein, 605 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRFLGGYVGGKAPKGFEWVSETKEITRNGRMVNVVINKLAHSTTPLTGPFEFEPDVIRWWWREIKTHKHLPLKPGSQAAIHPGSITGLCKRMDADAVPTRGETIGKKTASSAWDTGTVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGPMWIKDSYRCRKRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 11 - protein, 604 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRWLGGYVGGKAKKGFEYVSETKEITRNGRMVNVVINKLAHSTTPLTGPFEFEPDVIRWWWREIKTHKHLPFKPGSQAAIHPGSITGLCKRMDADAVPTRGETIGKKTASSAWDTGTVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMASKYYTAIQASYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 12 - protein, 605 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRQLGGYVGGKAGYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDEVLVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGPMWIKDSYRCRKRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 13 - protein, 605 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRFLGGYVGGKAPKGFEWVSETKEITRNGRMVNVVINKLAHSTTPLTGPFEFEPDVIRWWWREIKTHKHLPLKPGSQAAIHPGSITGLCKRMDADAVPTRGETIGKKTASSAWDTGTVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGSYVIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 14 - protein, 5 aa, synthetic construct
GGGGS

SEQ ID NO: 15 - protein, 10 aa, synthetic construct
GGGGSGGGGS

SEQ ID NO: 16 - protein, 15 aa, synthetic construct
GGGGSGGGGSGGGGS

SEQ ID NO: 17 - protein, 12 aa, synthetic construct
GSAGSAAGSGEF

SEQ ID NO: 18 - protein, 18 aa, synthetic construct
KESGSVSSEQLAQFRSLD

SEQ ID NO: 19 - protein, 14 aa, synthetic construct
EGKSSGSGSESKST

SEQ ID NO: 20 - protein, 22 aa, synthetic construct
GSAGSAAGSGEFGGGGSGGGGS

SEQ ID NO: 21 - protein, 30 aa, synthetic construct
GSAGSAAGSGEFKESGSVSSEQLAQFRSLD

SEQ ID NO: 22 - protein, 26 aa, synthetic construct
GSAGSAAGSGEFEGKSSGSGSESKST

SEQ ID NO: 23 - protein, 32 aa, synthetic construct
KESGSVSSEQLAQFRSLDEGKSSGSGSESKST

SEQ ID NO: 24 - protein, 1368 aa, Streptococcus pyogenes [sequence omitted]

SEQ ID NO: 25 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - DNA, 40 bp, synthetic construct
  source 1..40: mol_type = other DNA
gagcatcccaagaggactttcaggtctccttagaaaaccc

SEQ ID NO: 27 - DNA, 40 bp, synthetic construct
  source 1..40: mol_type = other DNA
gagcatcccaagaggacttgaaggtctccttagaaaaccc

SEQ ID NO: 28 - DNA, 1818 bp, synthetic construct
  source 1..1818: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagaggtggctgggcggctatgtgggcggcaaggccaagaagggcttcgaatatgtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgagaccatcggcaagaaaaccgccagcagcgcctgggacaccggcacggtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggctcctacgtgatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcctag

SEQ ID NO: 29 - DNA, 1818 bp, synthetic construct
  source 1..1818: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagaggttcctgggcggctatgtgggcggcaaggccccgaagggcttcgaatgggtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctctcaagcccggcagccaggccgccatccaccccggcagcatcaccggcctgtgtaagagaatggacgccgacgccgtgcccaccagaggcgagaccatcggcaagaaaaccgccagcagcgcctgggacaccggcactgtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcccgatgtggatcaaggacagctaccggtgccggaagagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcctag

SEQ ID NO: 30 - DNA, 1815 bp, synthetic construct
  source 1..1815: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagaggtggctgggcggctatgtgggcggcaaggccaagaagggcttcgaatatgtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgagaccatcggcaagaaaaccgccagcagcgcctgggacaccggcacggtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatggccagcaagtactacaccgctatccaggctagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcctag

SEQ ID NO: 31 - DNA, 1818 bp, synthetic construct
  source 1..1818: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagaggcagctgggcggctatgtgggcggcaaggccggctacggcttcgaacttgtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgagaccatcggcaagaaaaccgccagcagcgcctgggacgaagtgttggtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcccgatgtggatcaaggacagctaccggtgccggaagagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcctag

SEQ ID NO: 32 - DNA, 1818 bp, synthetic construct
  source 1..1818: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagaggttcctgggcggctatgtgggcggcaaggccccgaagggcttcgaatgggtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctctcaagcccggcagccaggccgccatccaccccggcagcatcaccggcctgtgtaagagaatggacgccgacgccgtgcccaccagaggcgagaccatcggcaagaaaaccgccagcagcgcctgggacaccggcactgtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggctcctacgtgatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcctag

SEQ ID NO: 33 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQREGARQIARKEWKEIGKIKEIKEGYLSLVIHEISKMVIKYNAIIVMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 34 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQREGARQIARKEWKEIGKIKEIKEGYLSLVIHEISKMVIKYNAIIVMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 35 - protein, 16 aa, synthetic construct
PKKKRKVEDPKKKRKV